(19) United States
(12) Patent Application Publication
(10) Pub. No.: US 2003/0191424 A1
(43) Pub. Date: Oct. 9, 2003

(54) Water resistant reusable sleeve for medical dressings and casts

(76) Inventor: Marc A. Skinner, Denver, CO (US)

Correspondence Address: Richard C. Litman, LITMAN LAW OFFICES, LTD., P.O. Box 15035, Arlington, VA, 22215, US

(21) Appl. No.: 10/114,996

(22) Filed: Apr. 4, 2002

(51) Int. Cl. A61B 19/12

(52) U.S. Cl. 602/62; 602/63; 128/856

(57) ABSTRACT

A water resistant reusable sleeve for medical dressings, inclusive of medical bandages and casts having a generally tubular member and defining a sleeve-like cavity tapered for receiving an extremity of an individual with a plaster cast, a splint, or a surgical dressing. The reusable sleeve is designed so as to be ergonomic or tapered to more closely accommodate the extremities of a user, providing a better fit, a more effective watertight seal with the skin, and also providing an important aesthetic advantage. Elastic bands of a size and elasticity so as to apply sufficient elastic force for sealing the sleeve to the appendage without creating a tourniquet effect are disposed on at least one end of the reusable sleeve. Furthermore, the water resistant reusable sleeve is preferably made of a composite material or a matrix material to prevent tearing of the material substrate during use and may incorporate a design.

### CROSS-REFERENCE TO RELATED APPLICATION

This application claims the benefit of U.S. Provisional Patent Application Serial No. 60/213,600, filed Jun. 22, 2000.

### BACKGROUND OF THE INVENTION

1. Field of the Invention

The present invention relates generally to limb protectors and more specifically to protective covers for medical bandages and casts.

2. Description of Related Art

Numerous apparatuses have been devised as protective coverings for casts or medical bandages. However, none of the limb protectors herein described presents a reusable protective cover for medical dressings which is ergonomically dimensioned and fabricated for reception of an extremity.

U.S. Pat. No. 4,562,834, issued to Bates et al., discusses a waterproof covering intended to be worn over a cast or bandage on an injured arm or leg. The covering comprises a generally tubular water impervious sleeve which is closed at one end and which has spaced cutting sites running from side to side along which the sleeve can be cut to different lengths. Apertured tabs are provided along the length of the covering to receive a strap for encircling the covering and sealing the covering around the limb, when the covering is cut at the cutting sites. Visual indicators identify the cutting sites. Additional seam segments serve to provide resistance to tearing at the side edges.

U.S. Pat. No. 5,063,919, issued to Silverberg, relates to a waterproof member for casts, splints, or other appliances or surgical dressings from water damage. A combination of a securing member and straps secures the member and provides a water proof seal between the waterproof member and the wearer.

U.S. Pat. No. 5,070,630, issued to Edmundson, discloses a decorative cast cover apparatus having a plurality of materials shaped to fit over a rigid cast on a patient and shaped in a three dimensional decorative shape and formed to hide the cast. The decorative cast cover is fastened to the cast with fastening members such as Velcro fasteners. A foam polystyrene is used to form a predetermined decorative shape, such as a shoe and sock, and a fabric material can be formed onto the polystyrene to provide a cast cover which looks like a shoe and sock covered foot and may have shoelaces passing through grommets in the fabric covering of the cast cover.

U.S. Pat. No. 5,395,302, issued to Botha et al., discloses a protective sheath for an injured limb, comprising an impervious sleeve made of a first plastics material, the sleeve having an open end through which a limb can be inserted. A sealing strip is fastened to and extends about the internal surface of the sleeve at the open end. The sealing strip is made of a second plastics material which is more highly plasticized than the first plastics material. There is also an elastic strap which is connected to and extends from the sleeve adjacent the open end. The strap is fastenable to itself by mating components of a self-contact fastener. In use with the sealing strip in contact with the limb, the strap can be stretched resiliently, wrapped about the open end and fastened to itself, thereby pressing the sealing strip against the limb to form a watertight seal at the open end.

U.S. Pat. No. 5,407,419, issued to Kelly et al., presents an enclosure for covering extremities or containing material such as trash or other objects or articles. The enclosure is a sack-like member having a lip extending above the opening or along a side edge of the sack-like member. The lip includes a perforation extending along a portion of the length thereof, parallel either to the opening or to the side edge. An adhesive strip with a removable covering may extend along the length of the lip at selected portions thereof. In use, the perforation is torn to separate the lip into a tie member and a securing member that remains attached to the sack-like member. In one embodiment, an extremity may be inserted into the sack-like member, and the tie member can be used to secure the opening around the extremity. In another embodiment, the tie member can be used to secure the opening around the extremity. In another embodiment, the tie member can be used to seal the opening of the sack-like member such that the enclosure can be used can be used as a container or receptacle in a desired application. When enclosing or covering an extremity, the enclosure protects a bandage or cast portion of the extremity from water. The enclosure may be sterilized to create a sterile environment within the sack member.

U.S. Pat. No. 5,439,439, issued to Green et al., discloses a method for applying orthopaedic bandages, The invention is a method of applying a material usable as an orthopaedic bandage which material is an open weave substrate having thereon a curable resin. The resin is curable, for example, by polymerization which is initiated by immersion of the resin-coated substrate in a bath. Gloves are worn during performance of the method which gloves are provided with a coating which in use is lubricous relative to the resin-coated substrate due to the substrate sticking to the gloves.

U.S. Pat. No. 5,592,953, issued to Delao, discusses a tubular sleeve with elasticized sealing means, including a protective sleeve for protectively covering an appendage and medicinal sites such as for intravenous equipment, bandages, wounds, etc. The sleeve is open at both ends with adjustable elastic sections which effectively seal the sleeve against the appendage. The elastic sections are equipped with elastic drawstrings which circumscribe a cushioning resilient layer. When the drawstrings are drawn and tied together the elastic force of the drawstring applies a sealing force to the resilient layer causing the layer to conform to the appendage configuration and seal the elastic section against the appendage. The wearer of the protective sleeve may safely shower and protect the medicinal site from contamination. When finished showering, the drawstrings may be untied to release the contractive force so as to permit the sleeve to be removed from the appendage.

U.S. Pat. No. 5,720,712, issued to Joy et al., relates to a re-usable limb protector for use in protecting a limb with a cast or bandage. The limb protector comprises stretchable moisture impervious material which is adjustable in size to accommodate wide range of individuals. The water tight seal is accomplished by stretching the moisture impervious base unit and is locked in place by the folding over of the two locking straps.

U.S. Pat. No. 4,139,003, issued to Little et al. disclose a flexible covering for placement over an individual's injured limb having a cast or bandage. The covering has a receptacle portion and a sealing portion which are designed respectively to receive the foot or hand of the limb and the leg or arm portion of the limb. The covering is designed for use by an individual with the injured limb when he is bathing in order to protect the cast or bandaged area from contact with water. The sealing portion has an interior diameter smaller than the diameter of the limb above the cast or bandage in order to provide a sealing engagement with the limb.

U.S. Pat. No. 4,301,603, issued to Scott, discusses a light weight flexible boot to be worn over a foot in a surgical cast and protect the cast from moisture. The boot has a walking sole of tough rubber type material with a cushion sole and an upper body, composed entirely of flexible and elastic foam material, with an inner fabric layer and an outer covering of water impervious fabric. The boot can be stretched over various sizes and shapes of casts and all joints in the boot are completely sealed and waterproof.

U.S. Pat. No. 5,761,746, issued to Brown, discloses a sleeve for keeping limbs dry with the lower section being composed of a waterproof material stretchable in longitudinal and latitudinal planes, and with the upper section being composed of a waterproof material exhibiting a substantial adherence characteristic, an including an elastic band for sealing-off the upper section when in use.

U.S. Design Patent No. 339,422, issued to Williams, illustrates a combined toe and cast cover. U.S. Design Patent No. 395,087, issued to Devries, depicts a medical cast cover. And, finally, U.S. Design Patent, issued to Rutledge et al., represents the ornamental design for a cast cover.

None of the above inventions and patents, taken either single or in combination, is seen to describe the instant invention a claimed.

### SUMMARY OF THE INVENTION

The water resistant reusable sleeve for medical dressings including casts, according to the invention, comprises a generally tubular member having a first circumferential end and a second circumferential end, the tubular member defining a sleeve-like cavity for receiving an extremity of an individual with a plaster cast, a splint, or a surgical dressing. The reusable sleeve is designed to help insure that wearers receive a proper ergonomic fit according to the shape and dimensions of their limbs, providing a more effective watertight seal with the skin, and also providing an important aesthetic advantage.

The reusable sleeve comprises components and materials for comfortable wear for as long as necessary, as wear of a device having an air tight abutment with the skin for extensive periods of time can result in excess pressure on the skin and loss of circulation. In the preferred embodiment of the invention, elastic bands of a size and elasticity so as to apply sufficient elastic force for sealing the sleeve to the extremity without creating a tourniquet effect are located on the material substrate of the second end and/or the first end of the invention. Additionally, Velcro fasteners, drawstrings, or any other fastener may be used to accomplish the intended purposes of providing an effective but comfortable watertight seal.

In the preferred embodiment of the invention, the second end of the reusable sleeve has a hand portion dimensioned and adapted to receive a hand. In an alternative embodiment, a foot portion may be integrally attached to the second end portion of the sleeve to form a covering for both the foot and lower limb. The water resistant reusable sleeve for medical dressings preferably comprises a composite material or a matrix material to prevent tearing of the material substrate during use. Additionally, the invention may comprise a non-porous membrane that is waterproof, windproof, and breathable.

Accordingly, it is a principal object of the invention to provide a water resistant reusable sleeve for medical dressings.

It is another object of the invention to provide a water resistant reusable sleeve for medical dressings to protect a cast for as long as necessary.

It is a further object of the invention to provide a water resistant reusable sleeve for medical dressings which can be worn during normal activities so that normal activity will not be curtailed.

Still another object of the invention is to provide a water resistant reusable sleeve for medical dressings which is ergonomic.

It is an object of the invention to provide improved elements and arrangements thereof in an apparatus for the purposes describes which is inexpensive, dependable and fully effective in accomplishing its intended purposes.

These and other objects of the present invention will become readily apparent upon further review of the following specification and drawings.

### BRIEF DESCRIPTION OF THE DRAWINGS

FIG. 1 is an environmental, perspective view of a water resistant reusable sleeve for medical dressings, according to the present invention.

FIG. 2 is a perspective, side view of the water resistant reusable sleeve for medical dressings, showing the manner of placement of a cast therein, according to the invention.

FIG. 3 is a side view of the water resistant reusable sleeve for medical dressings, according to the invention.

FIG. 4 is a side view of the water resistant reusable sleeve for medical dressings, showing the outline of a cast on the upper forearm and hand of a wearer, according to the invention.

Similar reference characters denote corresponding features consistently throughout the attached drawings.

### DETAILED DESCRIPTION OF THE PREFERRED EMBODIMENTS

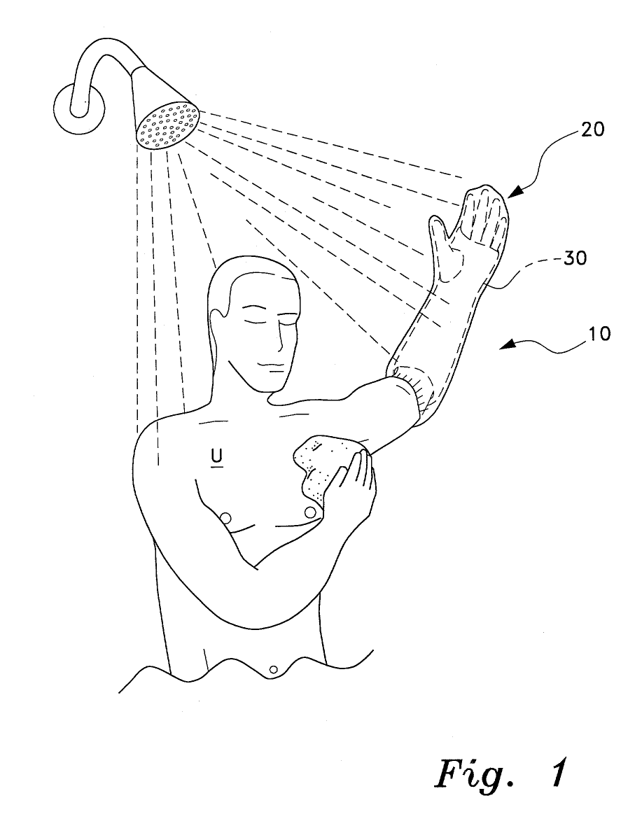

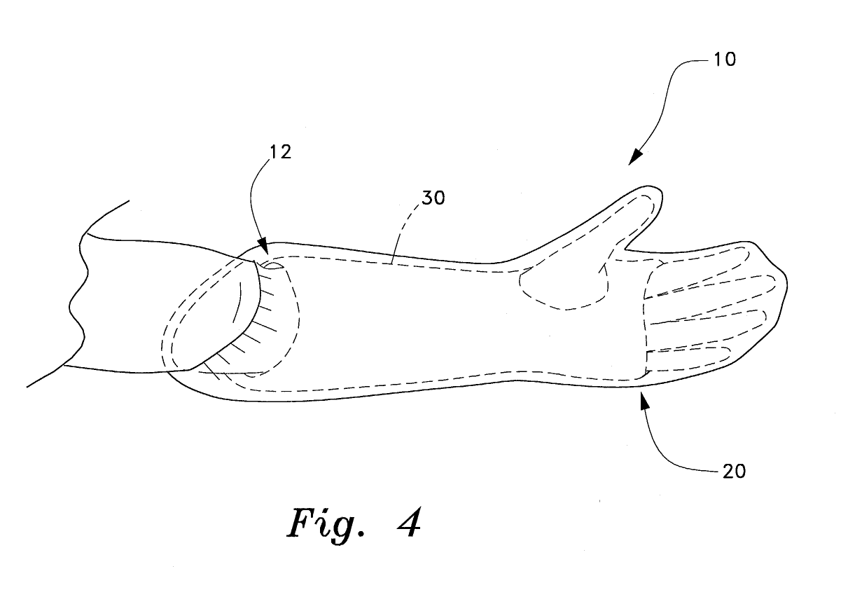

The present invention is directed to a water resistant reusable sleeve for medical dressings, inclusive of medical bandages and casts. The preferred embodiment of the invention is depicted in FIGS. 1 and 4 and is generally referenced by numeral 10.

For the purpose of the specification, the term medical dressing may be understood to comprise casts, bandages, or any other protective material covering or support for an injured or wounded site. Casts and bandages provide a critical part of the therapeutic regimen for broken and wounded limbs. However, medical dressings, especially casts and bandages, quickly loose their usefulness and form when exposed to excessive amounts of moisture, such as may occur during rain, bathing, and showering. Once this occurs, a patient is forced to return to his physician or nurse and have his cast or surgical dressing replaced, which can be an expensive and time-consuming undertaking. Even more dangerous is the fact that the loosening of a cast and the wetting of bandage can result in inadequate healing or infective processes being initiated. Moisture provides an excellent medium for the growth of fungi and bacteria, possibly leading to deleterious results.

The present invention provides a solution to this problem by introducing an elongated sleeve 10 adapted to be fitted over a limb having a cast, surgical dressing, or bandage disposed thereon. The invention is designed to be used with any medical bandage conventionally available, including adhesive bandages, flexible fabric bandages, rubber elastic bandages, gauze bandages, and triangular medical bandages. The reusable sleeve 10 is configured to comfortably fit over a cast and is preferably made of a matrix or composite material which is moderately stretchable so as to easily fit over casts, wrappings, and bandages. And while many limb protectors are made of stretchable moisture impervious material which is adjustable in size to accommodate users, the present invention is specially designed to be ergonomic or tapered so as to more closely accommodate the extremities of a user, also providing an important aesthetic advantage.

As shown in FIG. 1, the reusable sleeve 10 may be worn by user U during showering or bathing. The reusable sleeve is show covering the lower forearm and hand of a wearer, as well as the cast 30, indicated by ghost lines. In alternative embodiments of the invention, a design may be incorporated onto the outer surface of the sleeve 10, including designs related to popular culture, designs related to children's themes, and camouflage motifs for hunters and outdoorsmen.

A cast is generally defined as any device for mobilizing fracture area for a prolonged period of time. Half casts or splints are smaller versions of casts but can be adjusted to accommodate the swelling of tissue after injury. Splints may be made in a variety of shapes and sizes; however, most large casts are made of plaster materials and fiberglass, and include cotton and a protective material layer next to the skin.

The present invention comprises components and materials for wear as long as necessary, as wear of a device having an air tight abutment with the skin for extensive periods of time can result in trauma or debridement. Excess swelling below a cast or medical bandage may result in loss of circulation, loss of active movement of the toes and fingers, and pain. Thus, it is important that the reusable sleeve not be so tight when worn as to cause these symptoms.

Turning to the enlarged view in FIG. 4, in the preferred embodiment of the invention and as discussed further hereinbelow, an elastic band 12 is provided adjacent at least one end of the sleeve 10, whereby the wearer enjoys a comfortable, neat, and sufficiently close fit to prevent intrusion of moisture. The elastic band 12 must be sufficiently resilient to allow the sleeve to be reusable. The presence of the elastic band 12 cooperates with the material substrate of the sleeve 10 to prevent any inflow of soap, water, or any other deleterious substances.

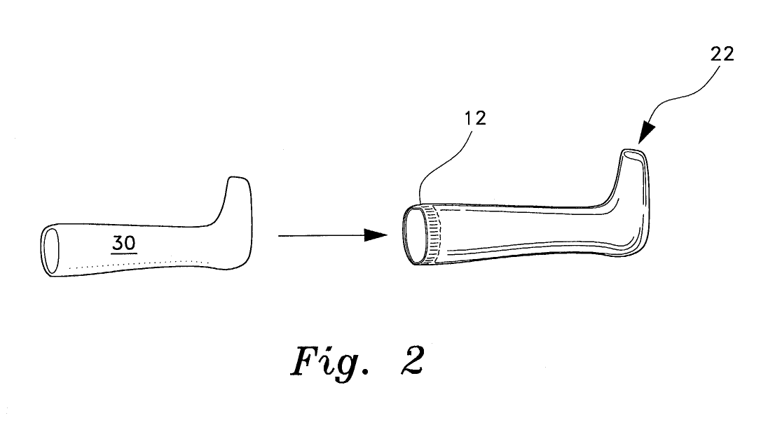

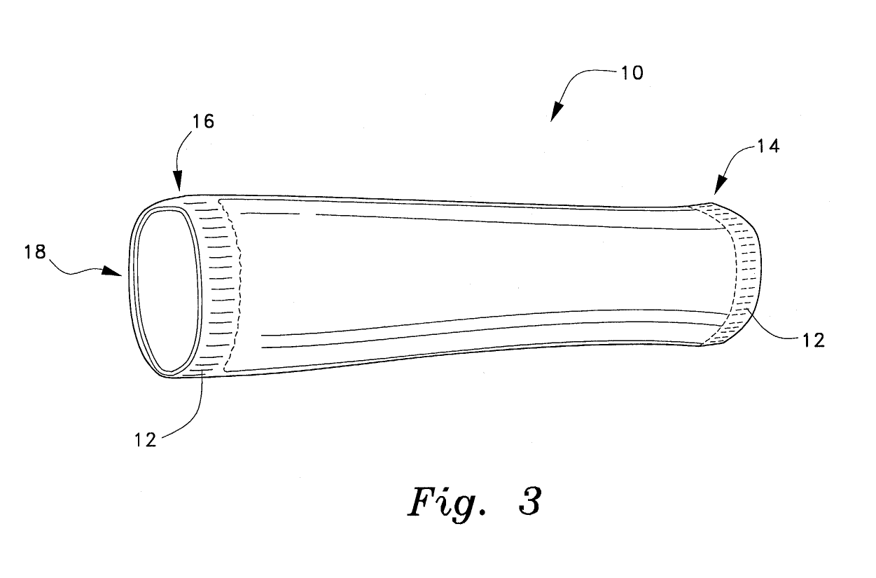

Turning to FIG. 3, in an alternative embodiment, it can now be fully appreciated that the reusable sleeve 10 comprises a generally tubular member having a first circumferential end 16 and a second circumferential end portion 14, the tubular member defining a sleeve-like cavity tapered for receiving an extremity of an individual with a plaster cast, a splint, or a surgical dressing, as indicated in FIG. 2; the first circumferential end 16 of the tubular member defines a first opening 18, the second circumferential end portion 14 similarly defines a second opening (not shown). Preferably, the first circumferential end 16 is greater in length than the second circumferential end, insuring that the wearer receives the proper, ergonomic fit of the invention according to the shape and dimensions of the injured limb.

Thus, in the alternative embodiment depicted in FIG. 3, bot the first end 16 and second end 14 further comprise at least one means for sealing-off their respective ends from water intrusion, which, as previously discussed, is preferably embodied as an elastic band 12. The means for sealing-off is sufficiently arranged so that the sealing portion has an interior diameter smaller than the diameter of the limb above the cast or bandage in order to provide a sealing engagement with the limb. The elastic bands 12 will preferably be installed during manufacture.

In the preferred embodiment of the invention, the elastic bands 12 are incorporated into the material substrate of the second end 14 and/or the first end 16. Elastic bands 12 such as commonly used by tailors may be employed, the elastic bands 12 being suitably fabricated from braided rubber, polyester, or other fibers. Additionally, Velcro fasteners, drawstrings, or any other fastener may be used to accomplish the intended purposes of the invention. The elastic bands 12 may be incorporated into the material substrate by folding the material substrate over the band and sewing, heat sealing, attaching by adhesive or by any other convenient method. Adjustability of the reusable sleeve 10 may also be provided by one or more passage means by bifolding the sleeve 10 member at the sleeve opening to provide an accordion shaped pleated fold to define a channel intended for receiving a securing means therein. In this manner, the securing means may be securely held between folds of the material substrate of the invention. Alternatively, the tightness of the sealing-off mean around the extremity may be varied to accommodate varying thicknesses of the extremities to prevent water from leaking into the cavity.

As shown in FIGS. 1 and 4, in the preferred embodiment of the invention, the second end portion 14 of the reusable sleeve 10 may have attached thereon a hand portion 20 dimensioned and adapted to receive a hand. In a similar alternative embodiment, such as seen in FIG. 2, a foot portion 22 may be integrally attached to the second end portion 14. Use of either a foot portion 22 or a glove or hand portion 20 extending integrally from or as part of the reusable sleeve 10 may comprise configurations thereof adapted to the specific dimensions of a foot and hand, respectively. For example, individual finger portions may be included, allowing the wearer of a bandage to more fully utilize their hands.

The water resistant reusable sleeve 10 for medical dressings preferably is made of a composite material or a matrix material to prevent tearing of the material substrate during use. Additionally, the invention may be made of a non-porous ultra-thin membrane that is waterproof, windproof, and breathable. Alternative materials include tightly woven, high performance laminated fabrics, polyester microfibers, multilayer laminates, and barrier systems. Nevertheless, whatever material substrate is used should preferably have a high rate of water vapor transmission to allow any perspiration to evaporate. And finally, the material substrate from which the sleeve 10 is made should be sufficiently strong and durable so as to be used on multiple occasions and may be recyclable.

It should now also be clearly apparent from the features of the reusable sleeve 10, various embodiments of the invention may include an operational method for providing a unitary reusable sleeve 10 on a limb with a medical dressing, such as a cast 30, the sleeve 10 being a generally tapered tubular member, wherein the steps of the method comprise:

(a) placing the sleeve 10 over an extremity, and

(b) adjusting the sleeve 10 such that it properly cooperates to protect the medical dressing against water intrusion.

It is to be understood that the present invention is not limited to the sole embodiments described above, but encompasses any and all embodiments within the scope of the following claims.

I claim:

1. A reusable sleeve for covering a medical dressing on wounded area of the body and for keeping an extremity dry when exposed to water, comprising:
a generally tubular member having a first circumferential end and a second circumferential end portion, said tubular member defining a sleeve-like cavity adapted for receiving an extremity of an individual;
said first end of said tubular member defining a first opening and a first circumference, said second end portion of said tubular member defining a second opening and a second circumference, wherein said first circumference is greater than said second circumference;
said first end further comprising at least one means for sealing-off said first end when in use, said at least one sealing-off means cooperating to provide a sealing protection against water intrusion.

2. The sleeve of claim 1 wherein said at least one first end sealing-off means includes an elastic band, and encircling said first end of said sleeve.

3. The sleeve of claim 1 wherein said second end portion further comprises a foot receiving portion for cooperatively receiving a foot therein.

4. The sleeve of claim 1, wherein said second end portion further comprises a hand receiving portion for cooperatively receiving a hand therein.

5. The sleeve of claim 4, wherein said hand receiving portion further comprises a thumb receiving portion for cooperatively receiving a thumb therein.

6. The sleeve of claim 4, wherein said hand receiving portion further comprises a finger receiving portion for cooperatively receiving the fingers therein.

7. The sleeve of claim 2, wherein said reusable sleeve is made of a breathable waterproof material.

8. The sleeve of claim 7, wherein said material is polyester microfiber material.

9. The sleeve of claim 7, wherein said material is a multilayer laminate.

10. The sleeve of claim 7, wherein said elastic bands are incorporated into the material substrate by bifolding the material and then folding the material substrate over the band and sewing, heat sealing, attaching by adhesive or by any other convenient method.

11. A reusable sleeve for covering a medical dressing on a wounded area of the body and for keeping an extremity dry when exposed to water, comprising:
a generally tubular member having a first circumferential end, said tubular member defining a sleeve-like cavity adapted for receiving an extremity of an individual;
said first end of said tubular member defining a first opening and a first circumference.
said first end further comprising at least one means for sealing-off said first end when in use, said at least one sealing-off means cooperating to provide a sealing protection against water intrusion.

12. The sleeve of claim 11, wherein said first end sealing-off means includes an elastic band, and encircling said first end of said sleeve.

13. The sleeve of claim 11, wherein said generally tubular member has a second end portion, said second end portion being foot receiving portion sized and shaped for cooperatively receiving a foot therein.

14. The sleeve of claim 1, wherein said generally tubular member has a second end portion, said second end portion being a hand receiving portion sized and shaped for cooperatively receiving a hand therein.

15. The sleeve of claim 14, wherein said hand receiving portion further comprises a thumb receiving portion for cooperatively receiving a thumb therein.

16. The sleeve of claim 15, wherein said hand receiving portion further comprises a finger receiving portion for cooperatively receiving the fingers therein.

17. The sleeve of claim 12, wherein said reusable sleeve is made of a breathable waterproof material.

18. The sleeve of claim 17, wherein said material is polyester microfiber material.

19. The sleeve of claim 17, wherein said material is a multilayer laminate.

20. The sleeve of claim 19, wherein said elastic bands are incorporated into the material substrate by folding the material substrate over said band and sewing, heat sealing, attaching by adhesive or by any other convenient method.
